(19) United States
(12) Patent Application Publication
(10) Pub. No.: US 2024/0389978 A1
(43) Pub. Date: Nov. 28, 2024

(54) PROBE SEALING STRUCTURE AND ULTRASONIC PROBE

(71) Applicant: ULTRASOUND BIOTECHNOLOGY (SHANGHAI) CO., LTD., Shanghai (CN)

(72) Inventors: Weizhong TANG, Shanghai (CN); Minmin ZHOU, Shanghai (CN)

(21) Appl. No.: 18/419,483

(22) Filed: Jan. 22, 2024

Related U.S. Application Data
(63) Continuation of application No. PCT/CN2023/097967, filed on Jun. 2, 2023.

(30) Foreign Application Priority Data
May 26, 2023 (CN) 202310610415.X

(51) Int. Cl. A61B 8/00

(52) U.S. Cl. CPC A61B 8/4455; A61B 8/4494

(57) ABSTRACT

Provided herein are a probe sealing structure and an ultrasonic probe. The probe sealing structure includes an outer pipe and a flexible driving shaft. A locking connector is arranged at a proximal end of the outer pipe. A blocking part is arranged in the locking connector and divides an inner part of the locking connector into two cavities. The flexible driving shaft is arranged in the outer pipe and the locking connector in a penetrating manner. A rotary inner core is arranged at a proximal end of the flexible driving shaft, and is inserted into a through hole of the blocking part. N sealing parts are movably sleeved outside one side, close to the blocking part, of the rotary inner core, and N is a positive integer, so as to realize sealing between the two cavities by the sealing parts. According to the present invention, the sealing parts are arranged between the locking connector and the rotary inner core to achieve volume compensation, thereby realizing sealing between the locking connector and the rotary inner core, and further preventing leakage of a coupling agent.

### CROSS-REFERENCE TO RELATED APPLICATION

The present application claims priority to Chinese Patent Application No.202310610415X filed on May 26, 2023. The contents of the above application are incorporated herein by reference.

### TECHNICAL FIELD

The present invention relates to the technical field of medical equipment, in particular to a probe sealing structure and an ultrasonic probe.

### BACKGROUND

Bronchial ultrasound, a relatively recent technological advancement, employs an ultrasonic bronchoscope or a micro ultrasonic probe. Using these tools, it navigates through the trachea and bronchial passages, scanning and providing a clear visualization of each layer of the trachea or bronchial wall. This includes displaying adjacent tissue structures around the lumen, such as ultrasonic images of mediastinal lymph nodes. The ultrasonic products used in clinical settings can be broadly categorized into two types. The first type is the ultrasonic bronchoscope, equipped with an ultrasonic probe embedded with an optical fiber structure. It allows scanning in the long axis direction of the airway and can guide real-time needle aspiration biopsies. The second type is the radial bronchial ultrasonic probe, requiring entry into the airway through a bronchoscope's biopsy channel. This probe produces a 360-degree image perpendicular to the airway's axis.

An ultrasonic probe having an imaging function for use with an endoscope is called an endoscopic ultrasonic probe. The endoscopic ultrasonic probe drives a single array element transducer by a slender flexible driving shaft to be immersed, by 360 degrees, in a plastic outer pipe having a coupling agent and with a front section sealed. In a process of rotation, the endoscopic ultrasonic probe transmits ultrasonic pulses at a certain frequency and receives echoes. The echoes carry information of a human body tissue penetrated by the ultrasonic waves, a human body image can be generated by processing echo signals, and at the end, the image information is combined with the transmitting and/or receiving message to finally generate an image.

In the prior art, in order to facilitate daily use and transportation of an endoscopic ultrasonic probe, a use temperature range and a transportation temperature range will be set, which may involve a temperature change of tens of degrees centigrade. A thermal expansion coefficient of a widely-used coupling agent is much higher than that of plastic making an outer pipe and that of plastic or metal making a locking connector and a rotary inner core. In addition, an acoustic window (outer pipe) of a current common ultrasonic probe is fixedly connected with a base, as shown in FIG. 1, with the relative position fixed. If no volume compensation device is designed, a huge pressure will be generated in a cavity when temperature rises in an actual use process, which will easily lead to leakage of the coupling agent. Therefore, there is an urgent need for a volume compensation device for solving leakage of the coupling agent.

### SUMMARY

The present invention intends to provide a probe sealing structure and an ultrasonic probe. A sealing part is arranged between a locking connector and a rotary inner core to achieve volume compensation, thereby realizing sealing between the locking connector and the rotary inner core, and further preventing leakage of a coupling agent.

In order to achieve the above objectives, the present invention provides a probe sealing structure, including an outer pipe and a flexible driving shaft. A locking connector is arranged at a proximal end of the outer pipe. A blocking part is arranged in the locking connector and an inner part of the locking connector is divided into two cavities by the blocking part. The flexible driving shaft is arranged within the outer pipe and the locking connector in a penetrating manner. A rotary inner core is arranged at a proximal end of the flexible driving shaft, and is inserted into a through hole of the blocking part. N sealing parts are movably sleeved outside one side, close to the blocking part, of the rotary inner core, and N is a positive integer such that sealing between the two cavities by the sealing parts is realized.

Optionally, the sealing part is configured to engage with both an inner side wall of the locking connector and an outer side wall of the rotary inner core.

Optionally, the sealing part is configured to be in interference fit with the inner side wall of the locking connector and to engage with the outer side wall of the rotary inner core.

Optionally, the sealing part is configured to be in interference fit with both the inner side wall of the locking connector and the outer side wall of the rotary inner core.

Optionally, the number of the sealing parts is at least two, and a gap exists between two adjacent sealing parts so as to make the two adjacent sealing parts moveable with each other on the rotary inner core in an axial direction.

Optionally, the sealing part is a sealing ring.

Optionally, the sealing ring has an O-shaped cross section in an axial direction, so as to make the sealing ring more closely engage with the inner side wall of the locking connector and the outer side wall of the rotary inner core.

Optionally, the sealing ring has a round cross section in a radial direction, so as to reduce a contact area between the sealing ring and the outer side wall of the rotary inner core.

Optionally, the sealing part is made of an elastic material.

The present invention provides an ultrasonic probe, including an ultrasonic transducer and the probe sealing structure, wherein the ultrasonic transducer is connected to a distal end of the flexible driving shaft and is movably arranged within the outer pipe, and a cavity of the outer pipe can be filled with a coupling agent for conducting ultrasonic waves.

The present invention has the following beneficial effects:

N sealing parts are movably sleeved outside one side, close to the blocking part, of the rotary inner core for realizing sealing between the outer side wall of the rotary inner core and the inner side wall of the locking connector, thereby preventing the coupling agent in a right cavity of the blocking part from entering into a left cavity and preventing leakage of the coupling agent.

The present invention has simple and reasonable structural design, can prevent leakage of the coupling agent in processes of processing, transportation, storage and use, and the device is highly reliably and conveniently prepared and processed.

### BRIEF DESCRIPTION OF THE DRAWINGS

FIG. 1 is a schematic structural diagram of an ultrasonic probe in the prior art;

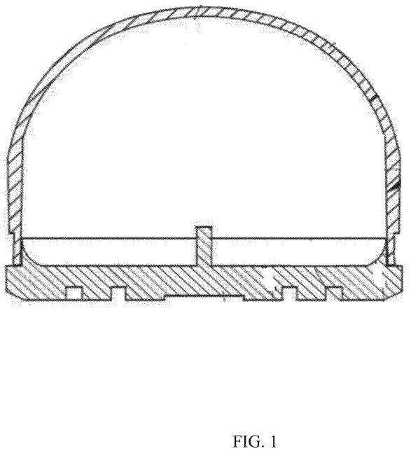

FIG. 2 is a schematic structural diagram of an embodiment in the present invention;

FIG. 3 is an enlarged schematic diagram of Structure A in FIG. 2 in the present invention;

FIG. 4 is a schematic structural diagram of a convex part in the present invention;

FIG. 5 is a schematic diagram of a matching structure of a convex part and a concave part in the present invention;

FIG. 6 is a schematic structural diagram of an ultrasonic probe in the present invention.

### REFERENCE NUMBERS IN THE DRAWINGS

1. Outer pipe; 11. Locking connector; 111. Blocking part; 1111. Through hole; 112. Cavity;
2. Flexible driving shaft; 21. Rotary inner core;
3. Sealing part; 31. Gap; 32. Sealing ring;
4. Ultrasonic transducer;
5. Convex part;
6. Concave part.

### DETAILED DESCRIPTION

In order to make the objectives, technical solutions and advantages of the present invention clearer, the technical solutions in embodiments of the present invention will be clearly and completely described below in conjunction with the accompanying drawings of the present invention. Obviously, the described embodiments are part of, but not all of, the embodiments of the present invention. Based on the embodiments of the present invention, all other embodiments obtained by those of ordinary skill in the art without creative work are within the protection scope of the present invention. Unless otherwise defined, technical or scientific terms used herein should be given their ordinary meanings as understood by those of ordinary skill in the art to which the present invention belongs. As used herein, the terms “comprise” and the like are intended to mean that an element or item appearing before the term encompasses elements or items appearing after the term and their equivalents, but does not exclude other elements or items.

It is worth noting that the term “distal end” as used herein refers to an end that extends into a patient's body and far away from an operator; and the term “proximal end” as used herein refers to an end that is close to the operator.

Sealing in the prior art can be divided into two categories: static sealing and dynamic sealing. Static sealing is mainly divided into three categories: gasket sealing, sealant sealing and direct contact sealing. According to working pressure, static sealing can be further divided into medium and low-pressure static sealing and high-pressure static sealing. Medium and low-pressure static sealing usually adopts a gasket which is soft and wide for sealing, and high-pressure static sealing, however, adopts a tough metal gasket with a narrow contact width. Dynamic sealing can be divided into two basic categories: rotary sealing and reciprocating sealing. In other conditions, contact sealing and non-contact sealing are divided based on whether a sealing piece is in contact with a part that moves relative to the sealing piece. Circumferential sealing and end face sealing are further divided based on a sealing piece and a contact position, and the end face sealing is further referred to as mechanical sealing. Centrifugal sealing and spiral sealing in dynamic sealing are sealing obtained when a machine operates to provide power for a medium, thereby being referred to as dynamic sealing sometimes. However, no sealing structure is arranged between an acoustic window (outer pipe) of a common ultrasonic probe and a base, a huge pressure will be generated in a cavity when temperature rises in an actual use process, which will easily lead to leakage of the coupling agent.

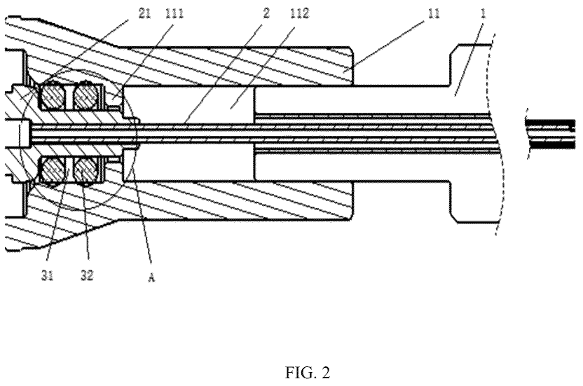

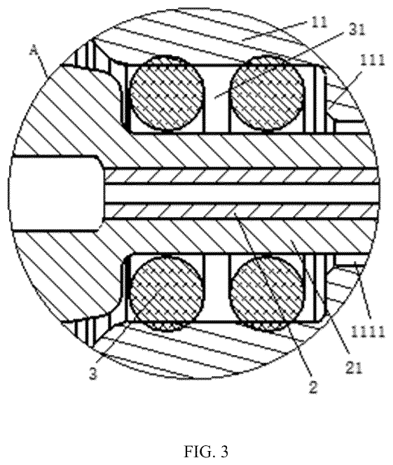

Aiming at the problems existing in the prior art, an embodiment of the present invention provides a probe sealing structure. FIG. 2 is a schematic structural diagram of an embodiment of the present invention. FIG. 3 is an enlarged schematic diagram of structure A in FIG. 2 in the present invention. The probe sealing structure includes an outer pipe 1 and a flexible driving shaft 2. A locking connector 11 is arranged at a proximal end of the outer pipe 1. A blocking part 111 (The blocking part can be understood as a blocking plate, the blocking plate can be welded in the locking connector 11, or the locking connector 11 can be integrally formed with the blocking plate) is arranged in the locking connector 11 and divides an inner part of the locking connector 11 into two cavities 112. The flexible driving shaft 2 is arranged in the outer pipe 1 and the locking connector 11 in a penetrating manner. A rotary inner core 21 is arranged at a proximal end of the flexible driving shaft 2, and is inserted into a through hole 1111 of the blocking part 111. N sealing parts 3 are movably sleeved outside one side, close to the blocking part 111, of the rotary inner core 21, and N is a positive integer, so as to realize sealing between the two cavities 112 (The two cavities are a left cavity and a right cavity with the blocking part 111 as a boundary, and at the beginning of work, a coupling agent is stored in the right cavity) by the sealing parts 3. A gap exists between an inner side wall of the locking connector 11 and an outer side wall of the rotary inner core 21, and the sealing part 3 is used for sealing the gap, thereby achieving a sealing effect.

When in use, the coupling agent is stored in the right cavity of the blocking part 111. In the prior art, as no volume compensation device is arranged between the locking connector 11 and the rotary inner core 21, a gap which makes the coupling agent to escape is easily generated between the locking connector 11 and the rotary inner core 21 under a condition of thermal expansion and cold contraction, making the coupling agent in the right cavity leak into the left cavity. In order to solve the above problem, in this embodiment, the sealing part 3 is arranged between the locking connector 11 and the rotary inner core 21 for compensating the volume and realizing sealing, thereby preventing the coupling agent from leaking from the right cavity to the left cavity and further preventing the coupling agent from leaking out of the device.

In a process of injecting the coupling agent into the outer pipe 1, an external drainage pipe is required for injection. Specifically, the drainage pipe is inserted to the outer pipe 1 and the coupling agent is then injected to the outer pipe 1 from the drainage pipe. As the coupling agent is heavier than the air, the air in the outer pipe 1 is pushed to be discharged outwards along with accumulation of the coupling agent at the distal end of the outer pipe 1. Along with accumulation of the coupling agent in the outer pipe 1, it is necessary to gradually pull out the drainage pipe. In the process of pulling out the drainage pipe, it is necessary to continuously inject the coupling agent to the outer pipe 1. With such operation, the air in the outer pipe 1 can be discharged. The flexible driving shaft 2 is then inserted to the outer pipe 1. In order to make the installation process faster and simpler, the ultrasonic transducer 4 is firstly installed at the distal end of the flexible driving shaft 2, thereby completing installation at one time, and improving the installation efficiency.

In one embodiment, the sealing part 3 is made of an elastic material, and the elastic material may be preferably a rubber material, but is not limited to the rubber material. As the rubber material has characteristics of thermal expansion and cold contraction, the volume of the sealing part 3 can be increased or decreased with temperature rise or drop, so as to ensure that the sealing part 3 can always block the gap.

In one embodiment, when the sealing part 3 is movably sleeved outside the rotary inner core 21 and assembled in the locking connector 11, the sealing part 3 and the inner side wall of the locking connector 11 and the outer side wall of the rotary inner core 21 have the following several position implementation modes.

In the first implementation mode, the sealing part 3 is configured to engage with both the inner side wall of the locking connector 11 and the outer side wall of the rotary inner core 21. In this implementation mode, when the rotary inner core 21 is static relative to the locking connector 11 (that is, when the rotary inner core 21 does not work), as the sealing part 3 is configured to engage with both the inner side wall of the locking connector 11 and the outer side wall of the rotary inner core 21, blocking of the gap can be realized by the sealing part, thereby achieving the sealing effect. When the rotary inner core 21 rotates relative to the locking connector 11 (that is, when the rotary inner core 21 works), the rotary inner core 21 may generate heat due to continuous rotation, the sealing part 3 expands when heated, making the sealing part 3 more closely engage with the inner side wall of the locking connector 11 and the outer side wall of the rotary inner core 21 and achieving a better sealing effect. When the rotary inner core 21 rotates to work, the rotary inner core 21 rotates relative to the sealing part 3, which will cause wear at a contact position between the sealing part 3 and the rotary inner core 21. However, due to temperature rise of the sealing part 3, the volume of the sealing part 3 will be increased under heat, making the sealing part 3 always contact or squeeze the rotary inner core 21 and always achieving a sealing effect.

In the second implementation mode, the sealing part 3 is configured to be in interference fit with the inner side wall of the locking connector 11 and is arranged in contact with the outer side wall of the rotary inner core 21. The difference between this implementation mode and the first implementation mode lies in that the sealing part 3 is configured to be in interference fit with the inner side wall of the locking connector 11, thereby better realizing sealing between the sealing part 3 and the locking connector 11.

In the third implementation mode, the sealing part 3 is configured to be in interference fit with both the inner side wall of the locking connector 11 and the outer side wall of the rotary inner core 21. Compared with the second implementation mode and the first implementation mode, the third implementation mode can achieve better sealing. However, as the sealing part 3 is in interference fit with the outer side wall of the rotary inner core 21, a contact area between the two is increased, and a friction force between the two is increased. In this way, when the rotary inner core 21 is operated to rotate, the operation process is extremely inconvenient due to the large friction force. Therefore, a person skilled in the art can set a specific implementation mode for positions between the sealing part 3 and the inner side wall of the locking connector 11 and the outer side wall of the rotary inner core 21 according to actual needs.

It should be noted that, whether in a low-temperature transportation environment or in a high-temperature transportation environment, the sealing part 3 is configured to engage with the inner side wall of the locking connector 11 and the outer side wall of the rotary inner core 21, and furthermore, interference fit can be set, so as to ensure that the device has a good sealing effect.

In one embodiment, as shown in FIG. 2 or FIG. 3, the number of the sealing parts 3 is at least two, and the gap 31 exists between two adjacent sealing parts 3, so as to make the two adjacent sealing parts 3 move with each other on the rotary inner core 21 in an axial direction. When in use, the expanded coupling agent will pass over the through hole 1111 of the blocking part 111 and enter into the left cavity, and push the rightmost sealing part 3 to move leftward in the gap 31. The existence of the gap 31 can alleviate the volume of the expanded coupling agent, thereby preventing the device from being damaged or the coupling agent from leaking due to excessive pressure. It is worth noting that when the rightmost sealing part 3 moves leftward in the gap 31, air in the gap 31 will be squeezed, and pressure will increase. In this way, as the temperature of the device drops, the coupling agent contracts under cooling, the volume is reduced, and a leftward contact force towards the rightmost sealing part 3 is lost. At this time, the rightmost sealing part 3 moves rightwards under pushing of compressed air in the gap 31, and the coupling agent entering into the left cavity is pushed into the right cavity, thereby achieving a better leak-proof effect.

In the implementation process, the number of the sealing parts 3 can be set to be one, two, three or more. It is worth noting that the more the number of the sealing parts 3 is, the better the sealing effect is, however, with increase of the number of the sealing parts 3, the contact or engagement area between the sealing parts 3 and the outer side wall of the rotary inner core 21 is larger, and the friction force is larger, which is adverse to rotation adjustment of the rotary inner core 21. In an example of FIG. 2 or FIG. 3, the number of the rotary inner core 21 is set to be two.

In one embodiment, the sealing part 3 is a sealing ring 32. The sealing ring 32 has an O-shaped cross section in an axial direction, so as to make the sealing ring 32 more closely attached to the inner side wall of the locking connector 11 and the outer side wall of the rotary inner core 21, as shown in FIG. 3. Arrangement of the O-shaped structure can further achieve a more convenient processing effect.

In one embodiment, the sealing ring 32 has a round cross section in the axial direction, so as to reduce a contact area between the sealing ring 32 and the outer side wall of the rotary inner core 21. The round structure is a preferable implementation mode and the implementation mode is not limited to the round structure. As shown in FIG. 3, the round structure not only makes the inner ring of the sealing ring 32 contact with the outer side wall of the rotary inner core 21 for sealing, but also reduces the contact area between the inner ring of the sealing ring 32 and the outer side wall of the rotary inner core 21 during contact, thereby reducing the friction force, and making rotation adjustment of the rotary inner core 21 relative to the locking connector 11 more convenient.

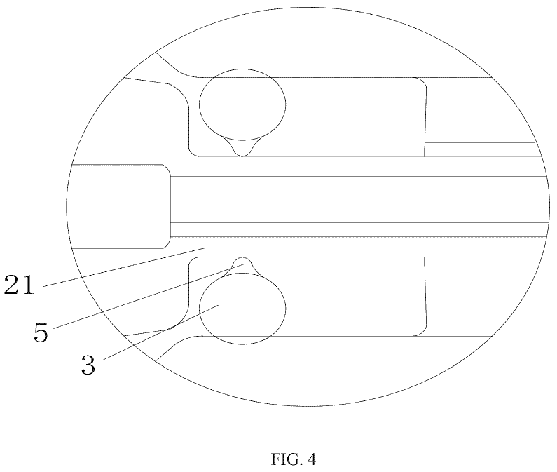

FIG. 4 is a schematic structural diagram of a convex part in the present invention. As shown in FIG. 4, the inner ring wall of the sealing ring 32 extends toward the axial center of the sealing ring 32 to form a convex part 5, and the convex part 5 abuts against the outer side wall of the rotary inner core 21. Preferably, the convex part 5 is in a ring-shaped structure, and the convex part 5 has a V-shaped cross section in a radial direction. One end, close to the sealing ring 32, of the V-shaped structure is larger than one end far away from the sealing ring 32 (it can be understood that a size of one end, far away from the sealing ring 32, of the convex part 5 is much smaller than a diameter of the sealing ring 32 in a round structure). In this way, a good sealing effect can be achieved between the sealing ring 32 and the outer side wall of the rotary inner core 21, and at the same time, the contact area between the sealing ring 32 and the rotary inner core 21 can be reduced and the friction force is reduced. In one embodiment, the convex part 5 is made of an elastic material, preferably a rubber material.

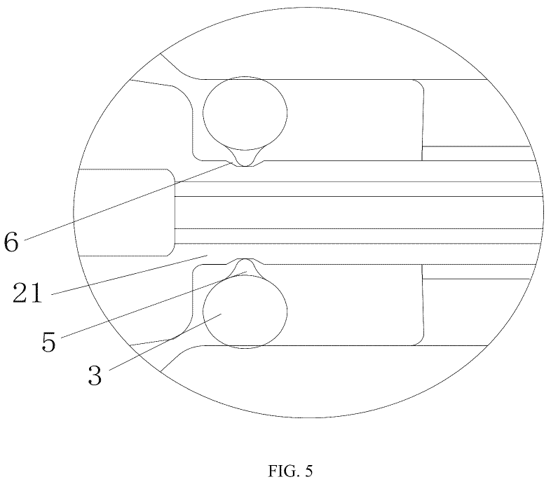

FIG. 5 is a schematic diagram of a matching structure of the convex part and a concave part in the present invention. As shown in FIG. 5, a concave part 6 matched with the convex part 5 is arranged on the outer side wall of the rotary inner core 21, and after assembling, the convex part 5 is movably arranged in the concave part 6. In this way, by matching between the convex part 5 and the concave part 6, a better sealing effect can be achieved. At the same time, due to existence of the concave part 6, the convex part 5 can be prevented from being compressed and deformed, thereby reducing the contact area between the convex part 5 and the outer side wall of the rotary inner core 21, reducing the friction force between the two and making rotation adjustment of the rotary inner core 21 more convenient.

In one embodiment, the concave part 6 is a ring-shaped groove, the ring-shaped groove has a groove-shaped cross section in a radial direction, and a cavity size of the groove-shaped structure is larger than the size of the V-shaped structure of the convex part 5, so as to make the convex part 5 move in the concave part 6 in the axial direction of the rotary inner core 21, thereby providing a buffer space for the expanded coupling agent. Preferably, an inclined platform is arranged at a right side of the groove-shaped structure. In this way, when the sealing part 3 moves rightward under pushing of air pressure, the coupling agent in the concave part 6 can be pushed into the right cavity by the convex part 5. Besides, one end, far away from the sealing part 3, of the convex part 5 is in contact with a bottom wall of the ring-shaped groove. In this way, a better sealing effect can be achieved.

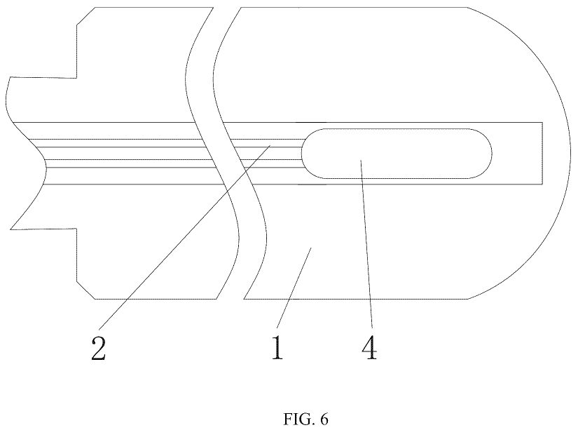

The present invention further provides an ultrasonic probe, including an ultrasonic transducer 4 and the probe sealing structure as described above, wherein the ultrasonic transducer 4 is connected to the distal end of the driving flexible shaft 2, the ultrasonic transducer 4 is movably arranged within the outer pipe 1, and the cavity of the outer pipe 1 can be filled with a coupling agent for conducting ultrasonic waves, as shown in FIG. 6.

While the implementation modes of the present invention have been described in detail above, it will be apparent to those skilled in the art that various modifications and variations can be made to the implementation modes. It is to be understood, however, that such modifications and variations are within the scope and spirit of the present invention as set forth in the claims. Moreover, the present invention described herein may have other implementation modes and may be implemented or realized in various ways.

1. A probe sealing structure, comprising an outer pipe and a flexible driving shaft, wherein a locking connector is arranged at a proximal end of the outer pipe; a blocking part is arranged in the locking connector and an inner part of the locking connector is divided into two cavities by the blocking part; the flexible driving shaft is arranged within the outer pipe and the locking connector in a penetrating manner; a rotary inner core is arranged at a proximal end of the flexible driving shaft and is inserted into a through hole of the blocking part; and N sealing parts are movably sleeved outside one side, close to the blocking part, of the rotary inner core, and N is a positive integer such that sealing between the two cavities by the sealing parts is realized.

2. The probe sealing structure of claim 1, wherein the sealing part is configured to engage with both an inner side wall of the locking connector and an outer side wall of the rotary inner core.

3. The probe sealing structure of claim 1, wherein the sealing part is configured to be in interference fit with an inner side wall of the locking connector and to engage with an outer side wall of the rotary inner core.

4. The probe sealing structure of claim 1, wherein the sealing part is configured to be in interference fit with both an inner side wall of the locking connector and an outer side wall of the rotary inner core.

5. The probe sealing structure of claim 1, wherein the number of the sealing parts is at least two and a gap exists between two adjacent sealing parts so as to make the two adjacent sealing parts movable with each other on the rotary inner core in an axial direction.

6. The probe sealing structure of claim 1, wherein the sealing part is a sealing ring.

7. The probe sealing structure of claim 6, wherein the sealing ring has an O-shaped cross section in an axial direction so as to make the sealing ring more closely engage with an inner side wall of the locking connector and an outer side wall of the rotary inner core.

8. The probe sealing structure of claim 6, wherein the sealing ring has a round cross section in a radial direction so as to reduce a contact area between the sealing ring and the outer side wall of the rotary inner core.

9. The probe sealing structure of claim 1, wherein the sealing part is made of an elastic material.

10. An ultrasonic probe, comprising an ultrasonic transducer and the probe sealing structure of claim 1, wherein the ultrasonic transducer is connected to a distal end of the flexible driving shaft and is movably arranged within the outer pipe, and a cavity of the outer pipe can be filled with a coupling agent for conducting ultrasonic waves.
